(19) United States
(12) Patent Application Publication
(10) Pub. No.: US 2003/0109920 A1
(43) Pub. Date: Jun. 12, 2003

(54) Engineered animal tissue

(75) Inventors: Manuela Martins-Green, Riverside, CA (US); QiJing Li, Riverside, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/012,194

(22) Filed: Dec. 6, 2001

(51) Int. Cl. A61F 2/10

(52) U.S. Cl. 623/1.41; 623/15.12; 623/912; 435/403

(57) ABSTRACT

An in vitro, three dimensional artificial tissue that resembles human skin has been developed. Microvascular endothelial cells from human adult lung were sandwiched between two layers of human dermal fibroblasts in three dimensional collagen gels. The sandwich was covered with keratinocytes. The cultures were self-maintained for prolonged periods of time without the addition of tumor promoters such as phorbol esters. Over a few days, the keratinocytes developed into a multilayered epithelium. Microvessels were produced in the support matrix. The microvessels were composed of a tight monolayer of endothelial cells surrounded by a continuous basal lamina, contacted by newly formed, sparse perioendothelial cells. The microvessels also contained newly formed blood cells. Human matrix molecules characteristic of skin were produced. This artificial tissue is an in vitro system that closely resembles human skin, and provides both a powerful model to study cellular and molecular mechanisms involved in skin development and replacement and a basis for a new generation skin replacement product.

### CROSS-REFERENCES TO RELATED APPLICATIONS

Not applicable.

### STATEMENT AS TO RIGHTS TO INVENTIONS MADE UNDER FEDERALLY SPONSORED RESEARCH AND DEVELOPMENT

Not applicable

### FIELD OF THE INVENTION

This invention pertains to the field of artificial animal tissue.

### BACKGROUND OF THE INVENTION

A major obstacle to studying cellular aspects of a variety of systems in humans is the lack of an appropriate and controllable model system. For example, it would be especially beneficial to have a system for studying angiogenesis. Angiogenesis is a crucially important component of embryonic development, normal physiological processes such as functions of the female reproductive tract, responses to traumatic events such a wound healing and recovery from ischemia, and pathological situations such as tumor development. Therefore, it is crucial to have an appropriate culture system that satisfactorily approximates the angiogenic environment in vivo. Studies of wound healing, cancer treatment, skin development, skin repair, skin replacement, and skin pharmatoxicology would also benefit from appropriate model systems.

Most studies to understand these types of biological processes have been conducted with animal models or with cell lines in two-dimensional cultures. Recently, three dimensional tissue culture systems have been developed that form endothelial cords with lumen (Black, et al., FASEB Journal (1998) 12:1331-1340). However, these systems are not self-maintaining and require the constant input of non-physiological factors such as phorbol esters (Montesano et al., 1983). Clearly a need exists for a three-dimensional human culture system that mimics connective tissue.

In addition, a need exists for autologous, in vitro reconstructed tissue for grafting to patients with severe trauma, e.g. nonhealing wounds or extensive burns. Early surgery and wound coverage by skin grafts have been shown to lead to better survival rates in such patients. Currently available sources of tissue for grafting include the classic options of cadaver skin and xenografts which have serious drawbacks including lack of permanence (Berthod, F. and Rouabhia, M. (1997) Exhaustive review of clinical alternatives for damaged skin replacement. In Skin Substitute Production by Tissue Engineering: Clinical and Fundamental Applications (Rouabhia, M., editor), pp 23-45, Landes Bioseciences, Austin). Recent attempts at grafting using engineered tissue have had limited success. Unpredictable viability due to delayed vascularization of the tissue is a likely problem (Berthod, F. and Damour, O., Br. J. Dermatol. (1997) 136:809-816; Boyce, S. T., et al., J. Invest. Dermatol. (1995) 104: 345-349; Young, D. M., et al.,. J. Burn Care Rehabil. (1996)17:305-310). Therefore, it would be beneficial to develop an engineered skin equivalent with functional vessels.

### SUMMARY OF THE INVENTION

The invention provides an artificial tissue including a support matrix, microvascular endothelial cells from a first animal, and connective tissue cells from a second animal, wherein the artificial tissue includes one or more microvessels produced by the artificial tissue. In preferred embodiments, the artificial tissue further includes epithelial cells from a third animal. Most preferably, the epithelial cells form a mutlilayered epithelium.

In one embodiment, the first, second and third animals from which the artificial tissue cells are derived are mammals. Preferably, the mammals are selected from the group consisting of primate, mouse, pig, cow, cat, goat, rabbit, rat, guinea pig, hamster, horse, or sheep. Most preferably, the mammals are humans. In one embodiment, the first, second and third animals are the same. In another embodiment, the first, second and third animals are different.

In one embodiment, the support matrix of the artificial tissue of the invention includes Vitrogen®. In another embodiment, the microvascular endothelial cells of the artificial tissue of the invention include primary human adult lung microvascular cells. In still another embodiment the connective tissue cells include primary adult human dermal fibroblasts. In a further embodiment, the epithelial cells of the artificial tissue include primary human adult keratinocytes. In one preferred embodiment, the invention provides an artificial tissue including Vitrogen®, primary human adult lung microvascular cells, and primary human dermal fibroblasts, wherein the artificial tissue includes one or more microvessels produced by the artificial tissue. In another preferred embodiment, the invention provides an artificial tissue including Vitrogen®, primary human adult lung microvascular cells, primary human dermal fibroblasts, and primary human keratinocytes, wherein the artificial tissue includes one or more microvessels produced by the artificial tissue.

In one embodiment, the artificial tissue of the invention is maintained in vitro. In a preferred embodiment, the artificial tissue of the invention is a composition suitable for tissue grafting.

The artificial tissue of the invention displays one or more characteristics of natural tissue. For example, the artificial tissue produces one or more compounds selected from the group consisting of laminin, fibronectin, collagen I, collagen III, hyaluronic acid, VEGF 145, VEGF 121, bFGF, IL-8, Syndecan-1, CXCR-1, CXCR-2, a mannose-containing protein, an acetylglucosamine-containing protein, PECAM-1, alpha-SMA, MMP-2, a growth factor receptor, plasminogen activator, mSRA, and CD68. In another embodiment, the microvessels of the artificial tissue produce one or more blood cells. In a preferred embodiment, the microvessels of artificial tissue produces one or more mononuclear leukocytes. In another embodiment, the artificial tissue produces one or more perioendothelial cells. In a further embodiment, the artificial tissue produces an extracellular matrix. In yet a further embodiment, the artificial tissue is self-maintained.

The invention also provides a method for producing an artificial tissue that entails mixing together a support matrix and connective tissue cells to form a support matrix-connective tissue mixture and forming a culture including two layers of support matrix-connective tissue mixture separated by a layer of endothelial cells, wherein the endothelial cells contact inner surfaces of the support matrix-connective tissue mixture layers. The invention includes the artificial tissue produced by this method. In a preferred embodiment, the above described method for producing an artificial tissue further includes plating a layer of epithelial cells on an outer surface of one layer of support matrix-connective tissue mixture. The invention includes the artificial tissue produced by said preferred method.

The invention further provides a method for studying a biological process, e.g., angiogenesis, wound healing, cancer treatment, skin development, skin repair, skin replacement, cell-cell interactions, cell-ECM interactions and skin pharmatoxicology. The method entails administering a test compound to the artificial tissue of the invention and measuring the effect of the test compound on a parameter of the biological process.

### BRIEF DESCRIPTION ON THE FIGURES

FIG. 1 is a schematic illustrating the three dimensional culture system.

FIG. 2 is a set of optical microscope pictures of three-dimensional culture cross sections, illustrating microvessels with lumen and perioendothelial cells. FIG. 2A is a section through a multilayered epithelium. FIG. 2B is a section through microvessels surrounded by numerous fibroblasts.

FIG. 3: The artificial tissue was treated with increasing concentrations of IL-8, and phosphorylation of the adhesive junction molecules PECAM-1 and VE-Cadherin was observed using immunoblots and antibodies to phosphor tyrosine (Anti-P-Y), PECAM-1 (AntiPECAM-1), and VE-Cadherin (Anti-VE-Cadherin).

FIG. 4: The artificial tissue was treated with increasing concentrations of IL-8, and production and activity of MMP-9 was measured in cell extracts and supernatant using gelatin zymogram assays.

FIG. 5: The artificial tissue was treated with increasing concentrations of IL-8, and production and activity of MMP-9, TIMP-1, and TIMP-2 were measured using immunoblot analysis.

FIG. 6: The artificial tissue was treated with increasing concentrations of IL-8, and activity of plasminogen activator was measured using an enzymatic assay specific for this enzyme.

### DETAILED DESCRIPTION

A novel artificial tissue has been created that produces microvessels. The artificial tissue is created using a support matrix, connective tissue cells, and microvessel endothelial cells to form a three-dimensional tissue culture that exhibits at least one characteristic of connective tissue. In further embodiments, the artificial tissue is created using a support matrix, connective tissue cells, microvessel endothelial cells, and epithelial cells to form a three dimensional tissue culture that exhibits at least one characteristic of skin tissue.

The artificial tissue forms one or more microvessels with a continuous basal lamina; this is an advantage of the invention. In various embodiments, these microvessels are characterized by any of a variety of structures and/or biochemical markers, e.g., the presence of perioendothelial cells, the presence of blood cells, the presence of endothelial cell specific proteins.

Another advantage of the invention is that the artificial tissue is stably maintained and therefore does not require the addition of non-physiological factors such as tumor promoters, e.g., phorbol esters and the like. An additional advantage of some embodiments of the artificial tissue is that the tissue may be created using primary cells. A further advantage is that the artificial tissue may be created using primary human cells. Due to these advantages, the artificial tissue is particularly useful for studying various biological processes, e.g., angiogenesis. In addition the artificial tissue is useful for tissue grafts for use in patients with skin trauma such as non-healing wounds or burns.

Definitions

Terms used in the claims and specification are defined as set forth below unless otherwise specified.

For the purposes of this invention, an “artificial tissue” is a non-naturally occurring composition including at least one cell type and a supporting material.

A “support matrix” is a supporting material suitable for use in cell culture. An example of a support matrix is Vitrogen (Cohesion Technologies, Inc.).

A “primary cell” is a cell taken directly from a living organism, which cell is not immortalized.

As used herein, the term “microvascular endothelial cells” refers to cells that are from, or are derived from (e.g., after passaging), the single layer of thin flattened cells that line blood vessels. One example of microvascular endothelial cells is human primary microvascular endothelial cells from lung.

The term “connective tissue cells” refers to cells that are from, or are derived from, connective tissue. Connective tissue is a mesodermally derived, fibrous tissue, and includes tissue which holds together the cells of an organ, and tissue which surround muscles and blood vessels, etc. In a preferred embodiment, connective tissue refers to tissue that rich in extracellular matrix and surrounds other more highly ordered tissues and organs. Examples of connective tissue cells include, but are not limited to, dermal fibroblasts.

The term “epithelial cells” refers to cells that are from or are derived from, epithelium. The epithelium is the covering of internal and external surfaces of the body, including the lining of body cavities and the skin. Examples of epithelial tissue cells include, but are not limited to, keratinocytes.

A “multilayered epithelium” is a structure that includes more than one layer of epithelial cells.

The term “microvessel” as used herein, refers to a tubular structure with one or more characteristics of any type of natural blood vessel (e.g., an artery, a vein, and the like) found in, for example, connective tissue. A microvessel can include a “junction” wherein a second microvessel has sprouted off of a first microvessel. As used herein, a microvessel includes a continuous basal lamina.

A “basal lamina” is sheet of extracellular matrix molecules characteristically found under and immediately adjacent to epithelial or endothelial cells. In one embodiment a basal lamina is characterized by the production of compounds such as collagen type IV, laminin, entactin, etc. A “continuous basal lamina” is intended to include an uninterrupted sheet of basal lamina that surrounds a microvessel as detected by, e.g., confocal microscopy visualization of a continuous sheet of anti-laminin antibodies bound to microvessels.

The term “extracellular matrix” or “ECM” refers to material with one or more characteristics of the non-cellular material secreted by connective tissue cells into the surrounding medium. Characteristic ECM material includes fibrous elements, e.g., collagen III (ColIII), link proteins, e.g., fibronectin (FN), and laminin (LN), and space filling molecules, e.g., glycosaminoglycans.

As used herein, the term “self-maintained” means that a cell culture survives and/or grows for prolonged periods of time (e.g., ten days or more) without the addition of additional non-physiological factors such as tumor promoters, e.g., phorbol esters and the like.

For the purposes of this invention, the term “blood cells” includes cells with one or more characteristics of cells from the blood stream, e.g., white blood cells, red blood cells, or platelets. These characteristics are well-known to one of skill in the art and may be morphological, physiological or biochemical.

The term “mononuclear leukocytes” refers to cells with one or more characteristics of mononuclear leukocytes, e.g., the cell expresses CD68. These characteristics are well-known to one of skill in the art and may be morphological, physiological or biochemical.

The term “perioendothelial cells” refers to cells with one or more characteristics of perioendothelial cells. These characteristics are well-known to one of skill in the art and may be morphological, physiological or biochemical. One example of said characteristics is expression of alpha smooth muscle actin (alpha-SMA).

As used herein, a biological “process” is an activity or function or series of activities or functions that is to be studied by a user. An example of a biological process is angiogenesis.

A “parameter” of a biological process is at least one aspect or feature of the biological process that can be observed or measure. For example, when studying the biological process of angiogenesis, one parameter that can be observed or measured is MMP-9 activity.

For the purposes of this invention, a “test compound” is any compound that is applied to the artificial tissue in order to examine the effect of the test compound on a parameter of a biological process. The compound may be natural or synthetic, purified or in a mixture.

One example of a test compound is the chemokine IL-8.

The term “suitable for tissue grafting” means that an artificial tissue is in a format that is appropriate for attempting transplantation or implantation into a living organism.

### DESCRIPTION OF THE PREFERRED EMBODIMENTS

The Artificial Tissue

As described in more detail below, a three dimensional artificial tissue is produced using a support matrix, connective tissue cells and microvascular endothelial cells.

The artificial tissue produces at least one microvessel that preferable include a continuous basal lamina. In one embodiment, the artificial tissue also includes epithelial cells which may form a multi-layered epithelium.

The cells used to produce the invention may be derived from any animal, including, but not limited to, non-mammalian vertebrates (e.g., avian, reptile and amphibian) and mammals. In a preferred embodiment, the animal cells are mammalian cells and are derived from a mammal including, but not limited to, a primate, a mouse, a pig, a cow, a cat, a goat, a rabbit, a rat, a guinea pig, a hamster, a horse, or a sheep. Primate cells are the preferred cells of the invention for many embodiments and include, for example, cells from humans, monkeys, orangutans, and baboons. In a preferred embodiment, the cells are human cells.

In one embodiment, the artificial tissue includes cells that are a mixture of cells from a plurality of species or animal. For example, the cells may be a mixture human and non-human cells. In one embodiment, the artificial tissue includes a mixture of human and primate cells. Alternatively, the artificial tissue includes cells that are all from the same species or animal. In a preferred embodiment, the cells are all human cells.

The cells used to produce the artificial tissue may be adult cells. Alternatively, the cells used may be non-adult cells, e.g. fetal, neonatal, umbilical, etc. Said cells may be all adult, or all non-adult, or a mixture thereof. In a preferred embodiment, the artificial tissue includes adult cells.

The cells can be from, or derived from normal tissue or abnormal tissue (i.e., tissue characterized by some physiological disorder, such as cancer). The cells can be primary cells or can be immortalized cells, e.g. altered cells with the ability to indefinitely reproduce. The cells may or may not be genetically altered. The preferred cells of the invention are primary cells from normal tissue.

To produce the artificial tissue, connective tissue cells are suspended in a support matrix to form a connective tissue-support matrix mixture. Examples of connective tissue cells suitable for use in the invention include dermal fibroblasts, pulmonary fibroblasts, mammary fibroblasts, bone marrow fibroblasts, etc . The choice of cell type is determined by the use of the tissue, e.g., which biological process is to be studied, and/or what type of tissue grafting is to be performed. In a preferred embodiment, the connective tissue cells are normal human dermal fibroblasts.

The support matrix is a supporting material suitable for cell cultures. A support matrix can include only natural components, only synthetic components, or both natural and synthetic components. In one embodiment, the support matrix includes carbon nanotubes to facilitate flow of electrical currents to facilitate wound healing. Tensile strength of the support matrix should be suitable for the intended application, e.g., a stronger support matrix is preferred for skin grafting. Examples of support matrices are well known to one of skill in the art and include bovine collagen I and III (e.g., Vitrogen (Cohesion Technologies, Inc.), and rat collagen. In a preferred embodiment the support matrix is Vitrogen.

The support matrix is generally formed by mixing the support matrix component(s) with a tissue culture medium suitable for growth of connective tissue cells, which includes a variety of commercially available media. Examples of such media are well known to one of skill in the art and include, e.g., 1×199 medium.

The artificial tissue is formed by co-culturing microvascular endothelial cells between two layers of the connective tissue-support matrix mixture described above. Examples of microvascular endothelial cells include but are not limited to mammary microvascular cells, umbilical microvascular cells, dermal microvascular cells, and lung microvascular cells. Again, the choice of cell type depends on the use of the tissue, e.g., which biological process is to be studied, and/or what type of tissue grafting is to be performed. In one embodiment the endothelial cells are adult human lung microvascular cells.

In a preferred embodiment, the artificial tissue is formed as follows. Vitrogen is prepared in 1×199 medium with 0.33 M NaOH at 4° C. Normal human dermal fibroblasts are evenly suspended by mixing the fibroblasts with the Vitrogen solution to form a homogeneous connective tissue-support matrix mixture. An aliquot of this connective tissue-support matrix mixture is applied to a tissue culture growth substrate, e.g., a tissue culture well, and incubated at 37° C. for 30 minutes. During this time, the mixture gels and forms the first layer of connective tissue-support matrix mixture. Primary microvascular endothelial cells from adult human lung are applied to the top of the first layer of connective tissue-support matrix mixture. Following incubation to allow attachment of the endothelial cells, a second layer of connective tissue-support matrix mixture is applied on top of the endothelial cells. An incubation period allows the second layer to gel.

In a further embodiment, the artificial tissue is formed by co-culturing microvascular endothelial cells between two layers of the connective tissue-support matrix mixture, as described above, and co-culturing epithelial cells on top of one layer of the connective tissue-support matrix mixture. Types of epithelial cells that may be used include, e.g., bladder epithelial cells, lung epithelial cells, stomach epithelial cells, and intestinal epithelial cells. The choice of cell type depends on the use of the tissue, e.g., which biological process is to be studied, and/or what type of tissue grafting is to be performed. In one embodiment, the epithelial cells are adult human keratinocytes. In a preferred embodiment, the artificial tissue is produced by co-culturing primary microvascular endothelial cells from adult human lung between two layers of a Vitrogen-primary adult normal human dermal fibroblast mixture, and co-culturing primary adult normal human keratinocytes on top of one layer of the Vitrogen-primary adult normal human dermal fibroblast mixture.

The artificial tissue of the invention can be grown in a variety of media that are well known to one of skill in the art. In particular, the culture medium contains appropriate nutrients and growth factors for the host cell employed. The nutrients and growth factors are, in many cases, well known or can be readily determined empirically by those skilled in the art. Suitable culture conditions for mammalian cells, for instance, are described in Mammalian Cell Culture (Mather ed., Plenum Press 1984); Barnes and Sato (1980) Cell 22:649; Mammalian Cell Biotechnology: a Practical Approach (Butler ed., IRL Press (1991). In one embodiment, more than one media is used at different stages of forming the artificial tissue, with an appropriate media used for each cell type, i.e., connective tissue, microvascular endothelial, and epithelial. In a preferred embodiment the media used include 1×EGM2-MV (Endothelial Growth Media 2, Microvascular, Clonetics), 1×FGM-2 (Fibroblast Growth Media 2, Clonetics), and IX KGM-2 (Keratinocyte Growth Media 2, Clonetics), etc. Supplements may or may not be used. In one embodiment, 5% FBS (fetal bovine serum) is included in the growth media. One of skill in the art will appreciate that the media used may be optimized for growth of different cell types.

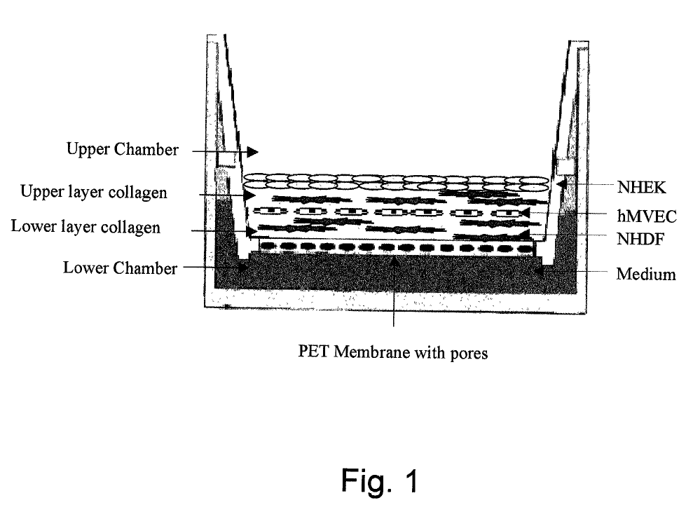

A wide range of formats can be used for growing the artificial tissue, including, but not limited to, cell culture bottles, flasks, dishes, and multi-well plates. An optimal parameter for the growth of epithelial cells, e.g., keratinocytes, is exposure to air. Therefore, in a preferred embodiment, the artificial tissue of the invention is grown using inserts, e.g., Transwell units, Biocoat cell culture control inserts, and the like as shown schematically in FIG. 1. In one embodiment, the artificial tissue is grown in 24 well plates with Biocoat cell culture control inserts (BD Biosciences). In another embodiment, the artificial tissue is grown in Transwell units (Becton Dickinson) with polyethylene tetraphthalate filters containing 8 micrometer pores.

In a preferred embodiment, the artificial tissue of the invention is self-maintaining, and as such does not require the addition of non-physiological compounds such as tumor promoters, e.g., phorbol esters.

Characterization of the Artificial Tissue

The artificial tissue of the invention comprises one or more structures and/or properties that are characteristic of natural tissue but have not been observed previously in artificial tissue. These structures and/or properties, as described in more detail below, can be identified by a wide range of techniques well known to one of skill in the art. The techniques may be morphological, physiological, and/or biochemical. Among the techniques are microscopy (electron, confocal, optical, Nomarski interference), immunolabelling, immunoblotting, northern blotting, ribonuclease protection assay (RPA), reverse transcriptase polymerase chain reaction, (RT-PCR), enzyme assays, e.g., enzyme linked immunoabsorbant assays (ELISAs), zymograms, and the like.

For example, one or more microvessels are produced in the artificial tissue of the invention. The microvessels are generally composed of a tight monolayer of endothelial cells surrounding a lumen. In one embodiment, the microvessels are contacted by sparse perioendothelial cells as observed by, e.g., optical microscopy. The perioendothelial cells can also be identified by cell specific markers. In one embodiment the perioendothelial cells are identified by the cell specific marker alpha-smooth muscle actin (alpha-SMA) using immunolabelling with alpha-SMA specific antibodies and confocal microscopy.

The microvessels produced by the artificial tissue of the invention are generally surrounded by a continuous basal lamina. Among the techniques that are used to detect the continuous basal lamina are electron microscopy to visualize the basal lamina morphology, confocal microscopy coupled to immunolabelling to detect basal lamina specific components (e.g., using anti lamina-specific antibodies), and biochemistry to isolate basal lamina specific molecules, etc. In one embodiment the continuous basal lamina is detected by confocal microscopy visualization of anti-laminin antibodies completely encircling microvessels of the artificial tissue.

In another embodiment, the microvessels present in the artificial tissue of the invention include junctions. The presence of the microvessel junctions can be confirmed using any of several microscopy techniques to visualize the microvessel morphology (e.g., phase contrast, Nomarski interference), immunolabelling to detect microvessel junction-specific components (e.g., Factor VIII), biochemistry to detect microvessel junction specific molecules such as platelet endothelial cell adhesion molecule (PECAM-1), etc. In a preferred embodiment the microvessel junctions are identified using immunoblots to identify production of PECAM-1.

The microvessels of the artificial tissue of the invention may also contain one or more blood cells. The cells may be identified by morphological methods, immunological methods, or both. In one example, the blood cells are identified by two markers for mononuclear leukocytes, CD68 and monocyte scavenger receptor (mSRA) using immunolabelling and confocal microscopy.

Another novel feature of the artificial tissue of the invention is the production of one or more compounds characteristic of dermal tissue. Among these types of compounds are ECM molecules, growth factors and cytokines, cell surface proteins, and the like. Among the ECM molecules that can be produced by artificial tissues of the invention are fibronectin (FN), collagen III (ColIII), collagen I (ColI), proteoglycans and hyaluronic acid. In one embodiment, the artificial tissue produces at least one of FN, ColIII, ColI, a proteoglycan, and hyaluronic acid. In another embodiment, the artificial tissue produces two or more of the following ECM molecules: FN, ColIII, ColI, a proteoglycan, and hyaluronic acid. In a further embodiment, the artificial tissue produces three or more of the following ECM molecules: FN, ColIII, ColI, a proteoglycan, and hyaluronic acid. In a still further embodiment, the artificial tissue produces four or more of the following ECM molecules: FN, ColIII, ColI, a proteoglycan, and hyaluronic acid. In a preferred embodiment, the artificial tissue of the invention produces all of the following ECM molecules: FN, ColIII, ColI, a proteoglycan, and hyaluronic acid.

Among the growth factors and cytokines that can be produced by the artificial tissue of the invention are VEGF 168, VEGF 145, VEGF 121, bFGF, EGF, PDGF, IGF, and interleukin-8 (IL-8). In one embodiment, the artificial tissue produces at least one of VEGF 168, VEGF 145, VEGF 121, bFGF, EGF, PDGF, IGF, and IL-8. In another embodiment, the artificial tissue produces at least two of VEGF 168, VEGF 145, VEGF 121, bFGF, EGF, PDGF, IGF, and IL-8. In still another embodiment, the artificial tissue produces at least three of VEGF 168, VEGF 145, VEGF 121, bFGF, EGF, PDGF, IGF, and IL-8. In a further embodiment, the artificial tissue produces at least four of VEGF 168, VEGF 145, VEGF 121, bFGF, EGF, PDGF, IGF, and IL-8. In a still further embodiment, the artificial tissue produces at least five of VEGF 168, VEGF 145, VEGF 121, bFGF, EGF, PDGF, IGF, and IL-8. In an alternative embodiment, the artificial tissue produces at least six of VEGF 168, VEGF 145, VEGF 121, bFGF, EGF, PDGF, IGF, and IL-8. In another alternative embodiment, the artificial tissue produces at least seven of VEGF 168, VEGF 145, VEGF 121, bFGF, EGF, PDGF, IGF, and IL-8. In a preferred embodiment, the artificial tissue produces all of the following: VEGF 168, VEGF 145, VEGF 121, bFGF, EGF, PDGF, IGF, and IL-8.

Among the cell surface proteins that can be produced by the artificial tissue of the invention are syndecan-1, CXCR-1, CXCR-2, cadherins, CAMs, growth factor receptors (e.g., bFGF receptor) and various mannose/n-acetylglucosamine containing proteins. In one embodiment, the artificial tissue produces at least one of syndecan-1, CXCR-1, CXCR-2, a cadherin, a CAM, a growth factor receptor, and a mannose/n-acetylglucosamine containing protein. In another embodiment, the artificial tissue produces at least two of syndecan-1, CXCR1, CXCR-2, a cadherin, a CAM, a growth factor receptor, and a mannose/n-acetylglucosamine containing protein. In still another embodiment, the artificial tissue produces at least three of syndecan-1, CXCR-1, CXCR-2, a cadherin, a CAM, a growth factor receptor, and a mannose/n-acetylglucosamine containing protein. In a further embodiment, the artificial tissue produces at least four of syndecan-1, CXCR-1, CXCR-2, a cadherin, a CAM, a growth factor receptor, and a mannose/n-acetylglucosamine containing protein. In a still further embodiment, the artificial tissue produces at least five of syndecan-1, CXCR-1, CXCR-2, a cadherin, a CAM, a growth factor receptor, and a mannose/n-acetylglucosamine containing protein. In an alternative embodiment, the artificial tissue produces at least six of syndecan-1, CXCR-1, CXCR-2, a cadherin, a CAM, a growth factor receptor, and a mannose/n-acetylglucosamine containing protein. In a preferred embodiment, the artificial tissue produces all of the following: syndecan-1, CXCR-1, CXCR-2, a cadherin, a CAM, a growth factor receptor, and a mannose/n-acetylglucosamine containing protein.

Uses of the Artificial Tissue

The artificial tissue of the invention is an excellent model for studying tissue development and repair, e.g. angiogenesis, wound healing, and tumor progression. Such studies can include evaluation of cell-cell interactions and cell-ECM interactions. The artificial tissue of the invention can also be used to study skin pharmacology, i.e., the sensitization and irritation of dermal tissue in response to a test compound.

In one embodiment, the artificial tissue of the invention is used to measure the effect of a test compound on any aspect of tissue development and repair. In a preferred embodiment, the artificial tissue of the invention is used to study angiogenesis. For example, the artificial tissue of the invention is used to study the role of cytokines in various aspects of angiogenesis including basal lamina integrity, cell adhesion, cell proliferation, cell migration, and microvessel tortuosity/sprouting. In an exemplary, preferred embodiment, the artificial tissue is used to study the role of the human chemokine IL-8 in basal lamina integrity. In a further, exemplary, preferred embodiment, the artificial tissue is used to study the role of the human chemokine IL-8 in basal lamina integrity by measuring the effect of IL-8 treatment on artificial tissue phosphorylation of VE-cadherin and PECAM-1, production and/or activity of Matrix Metalloproteinase 9 (MMP-9), and activation of Plasminogen Activator.

The artificial tissue of the invention can also be used to develop a new-generation tissue replacement product. For example, the artificial tissue may be used to treat a variety of skin trauma, including, e.g., nonhealing wounds. In one embodiment, the artificial tissue is produced in a format suitable for skin grafting. In a preferred embodiment, the artificial tissue is created using allogenic cells. Methods for testing and using tissue suitable for skin grafting can be found, e.g., in U.S. Pat. No. 5,512,475.

### EXAMPLE 1

### Formation of the Three Dimensional Artificial Tissue

A support matrix-connective tissue mixture was produced using the following steps. An interstitial support matrix of collagens I and III (Vitrogen from Cohesion Technologies) was prepared at 4° C., containing 10×199 medium, 0.33M NaOH and 3.0 mg/ml Vitrogen at a 2:1:17 ratio by volume. Primary adult normal human dermal fibroblasts (NHDF, Clonetics, Inc., Catalogue No. CC-25 11; Lot No: 16503) were passaged twice using standard tissue culture techniques. Briefly, the cells were plated at a density of 1×10⁶ cells/ml for 2 days at 37° C. and 5% CO₂ in normal plastic culture plates. The second passage of cells from the original stock were trypsinized as follows. Cells were washed twice with a glucose buffer (25 mM glucose, 140 mM NaCl, 5 mM KCl, 7 nM Na₂HPO4, pH 7.4). Trypsin (0.05% trypsin, 0.53 mM EDTA) was applied at RT. Cells were observed over time under the microscope to observe rounding up indicating release. The plates were tapped to release the single cells, and the trypsin was stopped with cold 1×media. The cells were centrifuged to a pellet (3000 rpm for 3 min) and resuspended in fresh 37° C. medium and then plated.

Using the trypsinized cells, 6×10⁴ NHDF cells (˜10 microliter volume in 1×FGM-2 medium, from Clonetics) were evenly suspended in 300 microliters of the collagen mixture and applied to either normal 24-well plates or 8 micrometer Biocoat cell culture control inserts (BD Biosciences). This first layer of support matrix-connective tissue mixture was incubated at 37° C. for 30 minutes.

After the mixture formed a gel, microvascular endothelial cells were applied as follows. Primary adult normal microvascular endothelial cells from human lung (hMVEC, Catalogue No. CC-2527; Lot No: 7F0629) were prepared as described above for connective tissue cells. After trypsin treatment, 1.5×10⁵ hMVEC in 300 microliters of 1×EGM2-MV medium (Clonetics) were applied on top of the gel and incubated for four hours at 37° C. Attachment of the hMVECs was confirmed by microscopic visualization.

The medium was removed and a second layer of support matrix-connective tissue mixture was applied on top of the hMVEC layer as described above and incubated for 30 minutes at 37° C. Finally, EGM2-MV medium was applied on top of the artificial tissue culture (1 ml of media in the 24 well plates, or 1 ml to the bottom chamber and 0.5 ml to the upper chamber in the Biocoat control inserts). Cultures were maintained at 37° C. and of 5% CO2 for at least 2 weeks. Medium was changed 24 hours after the cultures were started and then fed every 48 hours.

### EXAMPLE 2

### Formation of the Three Dimensional Artificial Tissue Resembling Skin

The artificial tissue as described in Example 1 was used as the starting material for producing an artificial tissue including epithelial cells. The artificial tissue was prepared using the Biocoat control inserts. Primary adult normal human epidermal keratinocytes (NHEK, Catalogue No. CC-2507; Lot No: 8F0538, Clonetics) were passaged twice as described above. The cells were applied (2×10⁵ NHEK in 500 microliters 1×KGM-2 medium (Clonetics) to the second layer of support matrix-connective tissue mixture. Media was added to both chambers of the Biocoat as follows: 1 ml of EGM2-MV was added to the lower chamber and 500 microliters of KGM-2 was added to the upper chamber. Media was changed 24 hours after the NHEK were added. This feeding was followed 48 hours later by removal of the KGM-2 media from the upper chamber to allow air exposure to the NHEK. The EGM2-MV in the lower chamber was changed every 48 hours.

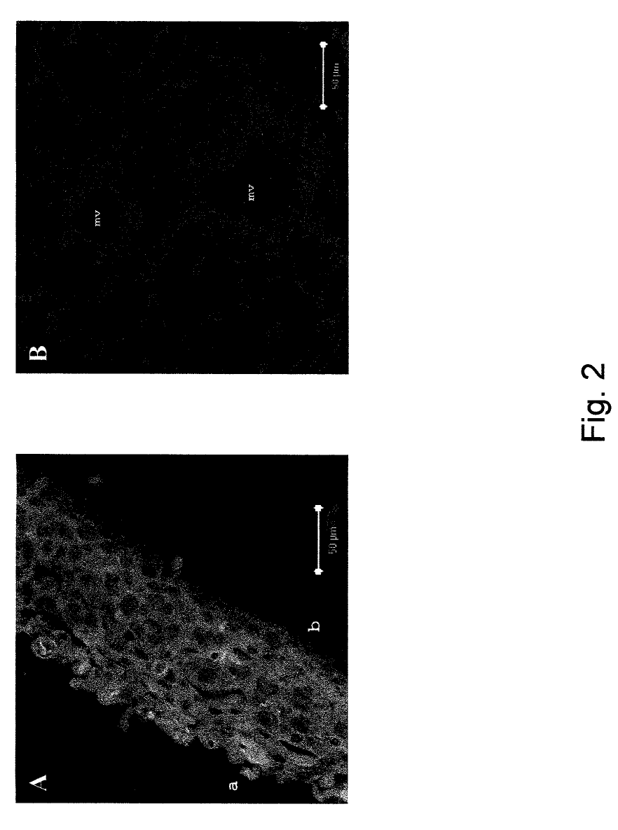

FIG. 2 shows representative pictures of the artificial tissue resembling skin, using optical microscopy and standard techniques (e.g., Wheater P R, Burkitt H G, Daniels V G (1987) Functional histology. A Text and Color Atlas. 2nd ed. Churchill Livingstone, New York). FIG. 2A is a section through a multilayered epithelium: the cell nuclei are blue and the cytoplasm is green. Like in real skin, the basal surface (b) contains the cells that divide, giving rise to the cells that progressively move up in the layer and eventually slough off at the apical surface (a). FIG. 2B is a section through microvessels surrounded by numerous fibroblasts.

### EXAMPLE 3

### Characterization of the Three Dimensional Artificial Tissue

The artificial tissue was characterized using a variety of standard techniques including microscopy and immunolabelling, northern blots, zymography, and enzyme assays.

Materials: All primary antibodies used for system characterization were specific for human antigen and were obtained from the following suppliers: anti-PECAM-1 (R&D system), anti-VE-Cadherin (Chemicon), anti-Laminin (Zymed), anti-Fibronectin (Santa Cruz Biotechnology), anti-Collagen III (Chemicon), anti-Tenascin (Chemicon), anti-Syndecan-1 (Serotec), anti-alpha-SMA (Sigma), anti-CD68 (DAKO), anti-mSRA (human macrophage scavenger receptor, kind gift from Dr. Motohito Takeya, Japan), anti-IL-8 (Intergen), anti-VEGF (Santa Cruz Biotechnology), anti-bFGF (Santa Cruz Biotechnology), anti-MMP-9 (Chemicon), anti-TIMPI (Chemicon), and anti-TIMP2 (Chemicon). Secondary antibodies and reagents used were: anti-mouse and anti-rabbit horseradish peroxidase (Amersham: Piscataway, N.J.), antirabbit and anti-mouse Alexa488 (Molecular Probes), anti-goat-FITC (Zymed) and anti-mouse Cy3 (Jackson ImmunoResearch). Other materials used include: biotinylated-HA binding protein (Seikagaku), WGA-Texas Red, ConA-Texas Red, Strepavidin-FITC, Strepavidin-Texas Red, nuclear staining dye TO-PRO3 (Molecular Probe), plasminogen activator substrate (CalBiochem), ECL reagents (Amersham), Vectashield mounting medium (Vector Laboratories), and DC protein assay kit (Bio-Rad).

Cells cultures: All cells, tissue culture media and materials for culture were purchased from Clonetics. Human primary microvascular endothelial cells (hMVEC), normal human dermal fibroblasts (NHDF) and normal human epidermal keratinocytes (NHEK) were used to create three dimensional artificial tissue cultures described above in Example 2. The cultures were set up in Transwell units (Biocoat control inserts, Becton Dickinson) with polyethylene tetraphthalate filters containing 8 μm pores. Cultures were maintained at 37° C. and of 5% CO₂ for at least 2 weeks.

Preparation of sections for immunolabelling: Ten-day-old cultures were fixed in 4% paraformaldehyde for 1-2 hours, washed in phosphate buffered saline (PBS) and incubated in 0.1M glycine-PBS for 10 minutes to quench free aldehyde groups. The samples were rinsed with PBS, and then incubated with 15% sucrose followed by 30% sucrose at 4° C. for cryoprotection. The specimens were embedded in Tissue Freezing Medium (Triangle Biochemical Sciences, Durham, N.C.) and frozen on dry ice. Each sample was cut into 10 micrometer to 25 micrometer sections and collected in gelatin-coated slides.

Immunolabelling of sections: Sections were rinsed with PBS to remove the Tissue Freezing Medium, refixed in 4% paraformaldehyde for 10 minutes, incubated in PBS containing 0.1M glycine for 10 minutes, followed by blocking with 10% non-immune serum of the species in which the secondary antibodies were generated. Primary antibodies were used at manufacturers recommended dilutions. Primary antibodies in 1% bovine serum albumin (BSA) in PBS were applied to the sections for 1 hour at room temperature (RT) or overnight at 4° C., washed extensively with 0.1% BSA/PBS. The sections were incubated in secondary antibody for 1 hour at RT. This was followed by staining with the nuclei dye TO-PRO3 (1:1000 dilution in PBS with 0.1% Triton-X100) at RT for 10-20 minutes. After extensive washes in PBS the sections were mounted in Vectashield.

Microscopy: Immunofluorescence was imaged using a Zeiss LSM510 microscope. 3D image reconstruction was performed with the LSM510 AIM software (Zeiss).

Protein sample preparation: Cultures used for protein extraction to perform immunoblots were prepared at 4° C. in IX RIPA (containing 300 mM NaCl, protease inhibitors and phosphatase inhibitor cocktail I & II (Sigma)). Cultures used for protein extraction to perform zymograms did not include inhibitors. The cultures from each well were released from the edge of culture insert and transferred to a 1.5 ml tube with 500 microliters extraction buffer with sterile forceps. The tubes were vortexed 10 times for one-half minute each time, with a 3 minute interval on ice between each vortexing step. Then tubes were left on a Nutator orbital shaker overnight at 4° C. The samples were centrifuged at 10,000 rpm for 10 minutes, and the supernatants were collected and stored at −70° C. until used.

Immunoblotting: SDS-PAGE was performed using 10% separating Doucet gels (Doucet, J., and J. Trifaro. A discontinous and highly porous sodium dodecyl sulfate-polyacrylamide slab gel system of high resolution. Anal. Biochem. (1988) 168:265-271.). Protein transfer to nitrocellulose membranes was performed using a wet-transfer apparatus (BioRad) at 100V for 45 min at room temperature. The membranes were blocked for 1 hour in 5% BSA in TTBS (0.05% Tween in Tris buffered saline) and incubated overnight at 4° C. in primary antibody diluted in 1% BSA in TTBS. The membranes were washed 3 times for 20 min each with TTBS, incubated in anti-mouse or anti-rabbit HRP (1:10,000) in 1% BSA for 1 hr, and washed 3 times for 20 minutes each with TTBS. The antibody bound to the membrane was visualized using ECL chemiluminescent kit (Amersham).

Zymography: Zymography was performed using 10% denaturing polyacrylamide gels as previously described (Paech and Christiansen. (1994) In Cell biology: a laboratory handbook. (J. Celis, Ed.) V.3 pp.264-271 San Diego: Academic Press). Briefly, gels to test for gelatinolytic activity contained 1% gelatin (Sigma) and were cast 12 hours before the sample separation. Protein samples were separated at 4° C. with 10 mA constant current for each gel. The proteins were renatured using two washes of 15 minutes each in 2.5% Triton-X100 twice at RT. Renaturation solution was replaced by developing buffer (50 mM Tris-HCl, 200 mM NaCl, 10 mM CaCi_{2, 0.02}% Brij 35, pH 7.5), the gels were incubated at 37° C. for 8-14 hours, fixed (methanol:water:acetic acid, 5:5:1) for 10 minutes, stained with 0.5% Coomassie Blue for 1 hour and destained (40% methanol plus 10% acetic acid) to obtain appropriate contrast.

Results:

Data and results are shown in Appendix A.

### EXAMPLE 4

### Using the Three Dimensional Artificial Tissue to Study Angiogenesis

The most common process by which new blood vessels form in adults occurs by sprouting from pre-existing vessels (angiogenesis). This process can be productively divided into the following steps: (1) Destabilization of endothelium, leading to blood vessel permeability and flexibility; (2) degradation of the basal lamina that surrounds the preexisting blood vessels; (3) localized proliferation of endothelial cells to provide cells for sprout formation; (4) endothelial cell migration, tube formation and elongation of the sprout; (5) stabilization of the endothelium by deposition of new basal lamina; (6) recruitment of perioendothelial support cells to the endothelium; (7) stabilization of the interactions between perioendothelial cells and endothelial cells; and (8) formation of mature microvessels. Numerous factors have been identified that participate in these steps of sprout formation during embryonic development. However, in adults, angiogenesis driven by injury or pathological conditions involves additional/alternative factors, including chemokines, of which IL-8 is a major player (e.g. Arenberg et al., 1997a & b).

It has been observed that the homologue of human IL-8 (hIL-8) in chickens, cCAF (chicken Chemotactic and Angiogenic Factor), stimulates angiogenesis in the Chorioallantoic Membrane (CAM) assay and in young chicks (Martins-Green and Feugate, 1998). Because of the high homology between cCAF and hIL-8 it is possible that the human chemokine also stimulates blood vessel sprouting. The artificial tissue of the invention was used to investigate the role of the test compound IL-8 on angiogenesis.

Cell cultures: Three dimensional artificial tissues were created as described above in Example 1 using primary normal human microvascular endothelial cells from adult lung (hMVEC; Clonetics), and primary adult normal human dermal fibroblasts (NHDF; Clonetics). The cultures were set up in either 24-well plates or Transwell units (Becton Dickinson) with polyethylene tetraphthalate filters containing 4 μm pores. Cultures were maintained at 37° C. and of 5% CO₂ for at least 2 weeks. Medium was changed 24 hours after the cultures were started and then fed every 48 hours.

Immunoblotting: SDS-PAGE was performed using 10% separating Doucet gels (Doucet, J., and J. Trifaro. A discontinous and highly porous sodium dodecyl sulfatepolyacrylamide slab gel system of high resolution. Anal. Biochem. (1988) 168:265-271.). Protein transfer to nitrocellulose membranes was performed using a wet-transfer apparatus (BioRad) at 100V for 45 min at room temperature. The membranes were blocked for 1 hour in 5% BSA in TTBS (0.05% Tween in Tris buffered saline) and incubated overnight at 4° C. in primary antibody diluted in 1% BSA in TTBS. The membranes were washed 3 times for 20 min each with TTBS, incubated in anti-mouse or anti-rabbit HRP (1:10,000) in 1% BSA for 1 hr, and washed 3 times for 20 minutes each with TTBS. The antibody bound to the membrane was visualized using ECL chemiluminescent kit (Amersham).

Zymography: Zymography was performed using 10% denaturing polyacrylamide gels as previously described (Paech and Christiansen. (1994) In Cell biology: a laboratory handbook. (J. Celis, Ed.) V.3 pp.264-271 San Diego: Academic Press). Briefly, gels to test for gelatinolytic activity contained 1% gelatin (Sigma) and were cast 12 hours before the sample separation. Protein samples were separated at 4° C. with 10 mA constant current for each gel. The proteins were renatured using two washes of 15 minutes each in 2.5% Triton-X100 twice at RT. Renaturation solution was replaced by developing buffer (50 mM Tris-HCl, 200 mM NaCl, 10mM CaCl_{2, 0.02}% Brij 35, pH 7.5), the gels were incubated at 37° C. for 8-14 hours, fixed (methanol:water:acetic acid, 5:5:1) for 10 minutes, stained with 0.5% Coomassie Blue for 1 hour and destained (40% methanol plus 10% acetic acid) to obtain appropriate contrast.

Plasminogen activity assay: A chromogenic plasminogen activator substrate (Boc-Val-Gly-Arg- NA.AcOH) was purchased from Calbiochem. 50 μl of a 10 mM stock solution of substrate in water was added to 200 μl of 0.1 mM HEPES buffer pH 8.0 and heated to 25° C. 25 μl cell extract was added to the substrate and absorbance was then read at 405 nm.

Results:

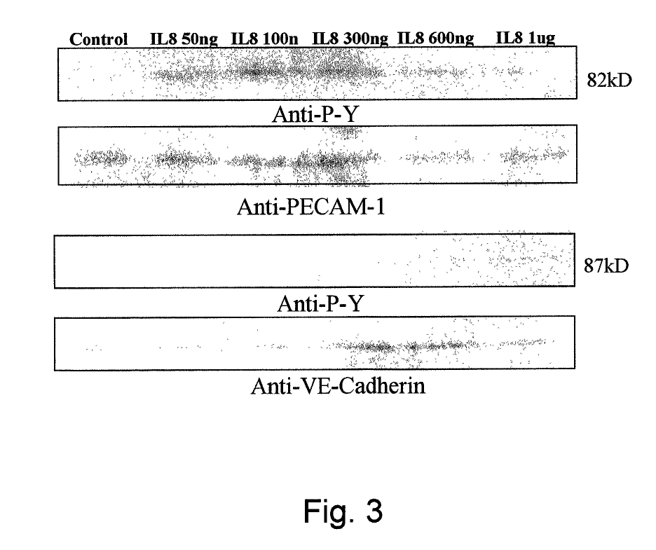

FIG. 3 illustrates the results from an experiment where the artificial tissue was treated with increasing concentrations of IL-8, and phosphorylation of the adhesive junction molecules PECAM-1 and VE-Cadherin was observed using immunoblots. The artificial tissue (one well in duplicate for each IL-8 concentration) was treated with IL-8 at the following concentrations (in ng/ml): 50, 100, 300, 600, and 1000. The samples were treated for 30 minutes and cell extracts were prepared, electrophoresed, and blotted. Membranes were probed with antibodies to phosphor tyrosine (Anti-P-Y), PECAM-1 (Anti-PECAM-1), and VE-Cadherin (Anti-VE-Cadherin). The results demonstrate that hIL-8 stimulates the phosphorylation of VE-cadherin and PECAM in microvessels. Therefore, IL-8 probably plays a role in destabilization of the endothelium reflected in loss of adhesion between the endothelial cells.

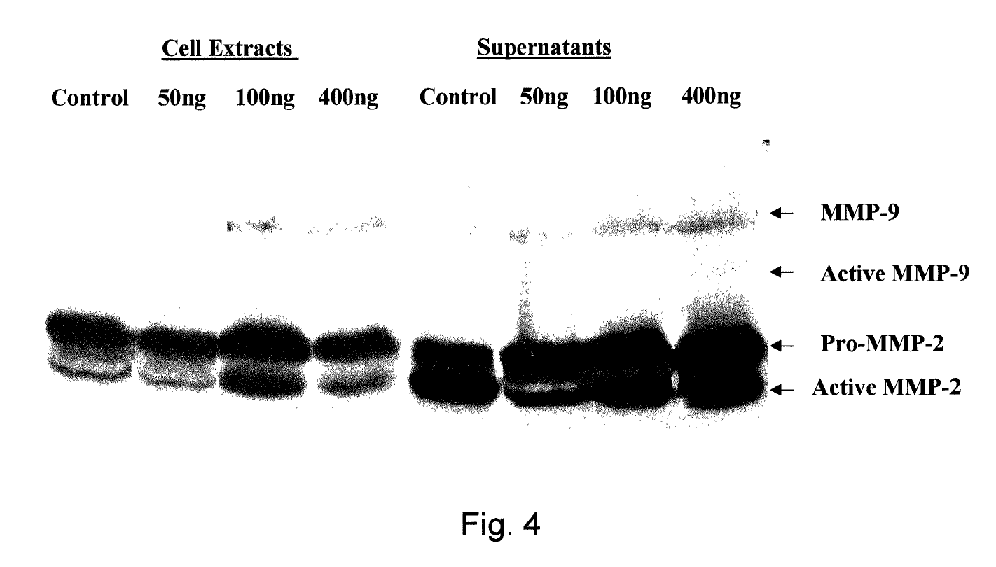

FIG. 4 illustrates the effects of IL-8 on production and activation of MMP-9 in the artificial tissue. Gelatin zymogram assays were performed to detect the production and activity of MMP-9. IL-8 increases MMP-9 production and activation in a dose-dependent manner. This effect was observed both in the Cell/ECM extracts and in the culture medium (supernatants). Samples were collected 24 hours after IL-8 treatment.

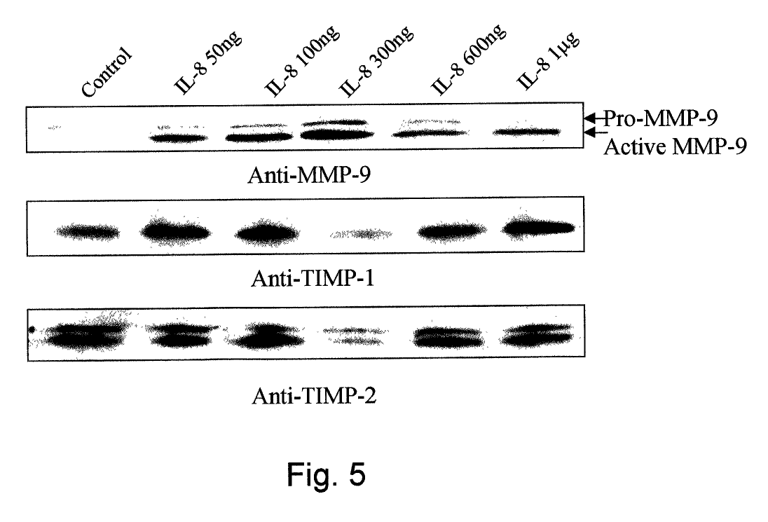

FIG. 5 illustrates the effects of IL-8 on MMP-9 and related natural tissue inhibitors. The artificial tissue was treated with increasing concentrations of IL-8, and production and activity of MMP-9 was measured using immunoblot analysis. The same blots were also probed for the MMP-9 natural tissue inhibitors TIMP-1 and TIMP-2. Samples were collected 3 hours after IL-8 treatment.

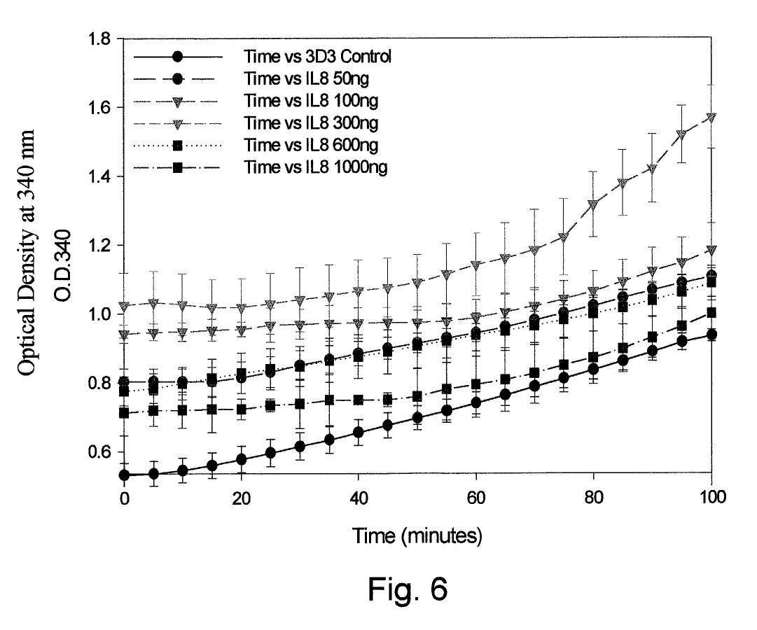

FIG. 6 illustrates the effects of IL-8 on activation of Plasminogen Activator. The artificial tissue was treated with increasing concentrations of IL-8, and activity of plasminogen activator was measured using an enzymatic assay specific for the enzyme.

Additional data and results are in Appendix B.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

### REFERENCES

Addison C L; Daniel T O; Burdick M D; Liu H; Ehlert J E; Xue Y Y; Buechi L; Walz A; Richmond A; Strieter R M. (2000) The CXC chemokine receptor 2, CXCR2, is the putative receptor for ELR+ CXC chemokine-induced angiogenic activity. Journal of Immunology, 165:5269-77.

Alattia J R; Kurokawa H; Ikura M. Structural view of adherin-mediated cell-cell adhesion. (1999). Cellular and Molecular Life Sciences, 55:359-67.

Alexander J S, Blaschuk O W and Haselton F R. (1992). An N-Cadherin-like protein contributes to solute barrier maintenance in cultured endothelium. J Cellular Physiol. 156:610-618.

Allt G; Lawrenson J G. (2001) Pericytes: cell biology and pathology. Cells Tissues Organs, 169:1-11.

Anastasiadis P Z; Reynolds A B. (2001) Regulation of Rho GTPases by p120-catenin. Current Opinion in Cell Biology, 13:604-10.

Arenberg D A; Polverini P J; Kunkel S L; Shanafelt A; Hesselgesser J; Horuk R; Strieter R M. (1997a) The role of CXC chemokines in the regulation of angiogenesis in non-small cell lung cancer. J Leuk Biol., 62:554-562.

Arenberg D A; Polverini P J; Kunkel S L; Shanafelt A; Strieter R M. (1997b) In vitro and in vivo systems to assess role of C-X-C chemokines in regulation of angiogenesis. Meth. Enzymol., 288:190-220.

Arenberg D A, Keane M P, DiGiovine B, Kunkel S L, Morris S B, Xue Y Y, Burdick M D, Glass M C, Iannettoni M D, Strieter R M (1998). Epithelial-neutrophil activating peptide (ENA-78) is an important angiogenic factor in non-small cell lung cancer. J Clin Investig 102:465-472.

Arihiro S; Ohtani H; Hiwatashi N; Torii A; Sorsa T; Nagura H. (2001) Vascular smooth muscle cells and pericytes express MMP-1, MMP-9, TIMP-1 and type I procollagen in inflammatory bowel disease. Histopathology, 39:50-9.

Ayalon O; Sabanai H; Lampugnani M G; Dejana E; Geiger B. (1994). Spatial and temporal relationships between cadherins and PECAM-1 in cell-cell junctions of human endothelial cells. J. Cell Biol. 126:247-58.

Baggiolini M, Dewald B, Moser B. (1994). Interleukin-8 and related chemotactic cytokines—CXC and CC chemokines. Adv Immunol 55, 97-179.

Baggiolini M; Dewald B, Moser B. (1997). Human chemokines: an update. Annual Review of Immunology, 15:675-705.

Balkwill F (1998). The molecular and cellular biology of the chemokines. J Viral Hepatitis 5:1-14.

Baluk, P, Bolton P, Hirata A, Thurston G, Fujiwara T, neal C R, Michel C C, McDonald, D M, 1997. Endothelial gaps: Time course of formation and closure in inflamed venules of rats. Am. J. Physiol 272: L155-L170.

Baluk, P, Hirata A, Thurston G, McDonald, D M, 1998. Endothelial gaps and adherent leukocytes in allergen-induced early- and late-phase plasma leakage in rat airways. Am J Pathol 152: 1463-1476.

Bartlett M R, Underwood P A, Parish C R (1995). Comparative analysis of the ability of leucocytes, endothelial cells and platelets to degrade the subendothelial basement membrane: evidence for cytokine dependence and detection of a novel sulfatase. Immunol Cell Biol 73:113-24.

Beck L Jr; D'Amore P A. (1997). Vascular development: cellular and molecular regulation. Faseb J, 11:365-73.

Belperio J A; Keane M P; Arenberg D A; Addison C L; Ehlert J E; Burdick M D; Strieter R M. (2000) CXC chemokines in angiogenesis. Journal of Leukocyte Biology, 68:1-8.

Bergers G; Brekken R; McMahon G; Vu T H; Itoh T; Tamaki K; Tanzawa K; Thorpe P; Itohara S; Werb Z; et al. (2000). Matrix metalloproteinase-9 triggers the angiogenic switch during carcinogenesis. Nature Cell Biology 2:737-744.

Bishop A L; Hall A. (2000) Rho GTPases and their effector proteins. Biochem Journal, 348 Pt 2:241-55.

Bleuel C C, Fuhlbrigge R C, Casasnovas J M, Aiuti A, Springer T A. (1996). A highly efficacious lymphocyte chemoattractant, stromal cell-derived factor 1 (SDF-1). J. Exp. Med. 184:1101-1109.

Bordon P, Heller R A (1997). Transcriptional control of matrix metalloproteinases and the tissue inhibitors of matrix metalloproteinases. Crit Rev Eukaryotic Gene Exp 7:159-178.

Brooks P C, Clark R A F, Cheresh D A. (1994). Requirement of vascular integrin v 3 for angiogenesis. Science 264:569-571.

Brooks P C; Stromblad S; Sanders L C; von Schalscha T L; Aimes R T; Stetler-Stevenson W G; Quigley J P; Cheresh D A (1996) Localization of matrix metalloproteinase MMP2 to the surface of invasive cells by interaction with integrin alpha v beta 3. Cell, 85:683-93.

Carmeliet P; Jain R K (2000) Angiogenesis in cancer and other diseases. Nature, 407:249-57.

Chang Y F; Novosel V; Chang C F. (1999) The isolation and sequence of canine interleukin-8 receptor. Dna Sequence, 10:183-7.

Corada M; Mariotti M; Thurston G; Smith K; Kunkel R; Brockhaus M; Lampugnani M G; Martin-Padura I; Stoppacciaro A; Ruco L; et al. (1999). Vascular endothelial-cadherin is an important determinant of microvascular integrity in vivo. Proc. Nat'l. Acad. Sci. USA, 96:9815-9820.

Corral C J, Siddiqui A, Wu L, Farrell C L, Lyons D, Mustoe T A. (1999) Vascular endothelial growth factor is more important than basic fibroblastic growth factor during ischemic wound healing. Archives of Surgery 134:200-205.

Coussens L M; Raymond W W; Bergers G; Laig-Webster M; Behrendtsen O; Werb Z; Caughey G H; Hanahan D. (1999). Inflammatory mast cells up-regulate angiogenesis during squamous epithelial carcinogenesis. Genes and Development, 13:1382-1397.

Coussens L M; Tinkle C L; Hanahan D; Werb Z. (2000). MMP-9 supplied by bone marrow-derived cells contributes to skin carcinogenesis. Cell, Oct 27, 103(3):481-90.

Davis G E; Pintar Allen K A; Salazar R; Maxwell S A. (2001) Matrix metalloproteinase-1 and -9 activation by plasmin regulates a novel endothelial cell-mediated mechanism of collagen gel contraction and capillary tube regression in three-dimensional collagen matrices. Journal of Cell Science, 114:917-30

Delarue F; Daunes S; Elhage R; Garcia A; Bayard F; Faye J. (1998) Estrogens modulate bovine vascular endothelial cell permeability and HSP 25 expression concomitantly. American Journal of Physiology, 275:H1011-5.

Dejana E, Corada M, Lampugnani M G. (1995). Endothelial cell-to-cell junctions. Faseb J, 9:910-918.

Dejana E; Lampugnani M G; Martinez-Estrada O; Bazzoni G. (2000) The molecular organization of endothelial junctions and their functional role in vascular morphogenesis and permeability. International Journal of Developmental Biology, 44:743-8.

Dunlevy J R; Couchman J R. (1995) Interleukin-8 induces motile behavior and loss of focal adhesions in primary fibroblasts. Journal of Cell Science, 108:311-21.

Dvorak A M; Kohn S; Morgan E S; Fox P; Nagy J A; Dvorak H F. (1996) The vesiculo-vacuolar organelle (VVO): a distinct endothelial cell structure that provides a transcellular pathway for macromolecular extravasation. Journal of Leukocyte Biology, 59: 100-15.

Eliceiri B P; Cheresh D A. (2000) Role of alpha v integrins during angiogenesis. Cancer Journal from Scientific American, 6 Suppl 3:S245-9.

Engelhardt E; Toksoy A; Goebeler M; Debus S; Brocker E B; Gillitzer R. (1998) Chemokines IL-8, GROalpha, MCP-1, IP-10, and Mig are sequentially and differentially expressed during phase-specific infiltration of leukocyte subsets in human wound healing. American Journal of Pathology, 153:1849-60.

Esser S; Lampugnani M G; Corada M; Dejana E; Risau W. (1998) Vascular endothelial growth factor induces VE-cadherin tyrosine phosphorylation in endothelial cells. J. Cell Science, 111:1853-65.

Fang, K, M Martins-Green, L T Williams, & H Hanafusa, (1996). Molecular cloning of chicken protein tyrosine phosphatase a and its expression in the central nervous system. Molecular Brain Research 37:1-14.

Feldman H, Bugany H, Mahner F, Klenk H D, Drenckhahn D, Schnittler H J (1996). Filovirus-induced endothelial leakage triggered by infected monocytes/macrophages. J Virology 70:2208-2214.

Feugate, J. E. and Martins-Green. M (2001). The CXC chemokine cCAF stimulates differentiation of fibroblasts into myofibroblasts and accelerates wound closure in vivo. J. Cell Biol. Accepted upon final revisions.

Friedlander M, Brooks P C, Shaffer R W, Kincaid C M, Vamer J A, and Cheresh D A. (1995). Definition of two angiogenic pathways by distinct αᵥ integrins. Science 270:1500-1502.

Garcia J G, Schaphorst K L, Verin A D, Vepa S, Patterson C E, Natarajan V. (2000) Diperoxovanadate alters endothelial cell focal contacts and barrier function: role of tyrosine phosphorylation. J. App. Physiol. 89:2333-2343.

Gharaee-Kermani M, Denholm E M, Phan S H. (1996). Costimulation of fibroblast collagen and transforming growth factor betal gene expression by monocyte chemoattractant protein-1 via specific receptors. J. Biol. Chem. 271:17779-17784.

Ghirniker R S, Lee Y L, Eng L F (1998). Inflammation in traumatic brain injury: role of cytokines and chemokines. Neurochem Res 23:329-340.

Grant D S, and Kleinman H K (1997). Regulation of capillary formation by laminin and other components of the extracellular matrix. in Regulation of Angiogenesis, eds Goldberg I D, and Rosen E M., pp. 317-333, Birkhauser Verlag, Basal, Switzerland.

Gumbiner B M. (2000) Regulation of cadherin adhesive activity. Journal of Cell Biology, 148:399-404.

Haas T L, Davis S J, and Madri J A. (1998). Three-dimensional type I collagen lattices induce coordinate expression of matrix metalloproteinases MT1-MMP and MMP-2 in microvascular endothelial cells. J. Biol. Chem. 273:3604-3610.

Haas T L, Madri J A (1999). Extracellular matrix-driven matrix metalloproteinase production in endothelial cells: implications for angiogenesis. Trends Cardiovasc Med 9:70-77.

Han Y P, Tuan T L, Hughes M, Wu H, Garner W L. (2001) Transforming growth factor-beta- and tumor necrosis factor-alpha-mediated induction and proteolytic activation of MMP-9 in human skin. J. Biol. Chem. 276:22341-22350.

Hanahan D. (1997). Signaling vascular morphogenesis and maintenance. Science 277:48-50.

Hildebrand J D; Soriano P. (1999) Shroom, a PDZ domain-containing actin-binding protein, is required for neural tube morphogenesis in mice. Cell, 99:485-97.

Hiraoka N, Allen E, Apel I J, Gyetko M R, Weiss S J (1998). Matrix metalloproteinases regulate neovascularization by acting as pericellular fibrinolysins. Cell 95:365-377.

Hirata A; Baluk P; Fujiwara T; McDonald D M. (1995). Location of focal silver staining at endothelial gaps in inflamed venules examined by scanning electron microscopy. Amer. J. Physiol., 269:L403-18.

Hirschi K K; D'Amore P A. (1996) Pericytes in the microvasculature. Cardiovascular Research 32:687-98.

Hofmann A, Nolan G P, Blau H M (1996). Rapid retroviral delivery of tetracycline-inducible genes in a single autoregulatory cassette. Proc Natl Acad Sci USA 28:5185-5190.

Horuk, R. (ed.) (1996). Chemoattractant Ligands and Their Receptors. 377 pp. (CRC Press, N.Y).

Howard O M; Ben-Baruch A; Oppenheim J J. (1996) Chemokines: progress toward identifying molecular targets for therapeutic agents. Trends in Biotechnology, 14:46-51.

Ikeda W; Nakanishi H; Miyoshi J; Mandai K; Ishizaki H; Tanaka M; Togawa A; Takahashi K; Nishioka H; Yoshida H; et al. (1999) Afadin: A key molecule essential for structural organization of cell-cell junctions of polarized epithelia during embryogenesis. Journal of Cell Biology, 146:1117-32.

Ilan N; Mahooti S; Madri J A. (1998). Distinct signal transduction pathways are utilized during the tube formation and survival phases of in vitro angiogenesis. J Cell Sci 111:3621-31.

Ilan N; Cheung L; Pinter E; Madri J A. (2000) Platelet-endothelial cell adhesion molecule-1 (CD31), a scaffolding molecule for selected catenin family members whose binding is mediated by different tyrosine and serine/threonine phosphorylation. Journal of Biological Chemistry, 275:21435-43.

Juremalm M; Hjertson M; Olsson N; Harvima I; Nilsson K; Nilsson G. 2000. The chemokine receptor CXCR4 is expressed within the mast cell lineage and its ligand stromal cell-derived factor-1 alpha acts as a mast cell chemotaxin. European J Immunol., 30:3614-3622.

Karasek M A (1999). Progress in our understanding of the biology of psoriasis. Cutis 64:319-322.

Keane M P, Strieter R M. (1999). The role of CXC chemokines in the regulation of angiogenesis. Chemical Immunology, 72:86-101.

Kim S, Bell K, Mousa S A, Varner J A. (2000a) Regulation of angiogenesis in vivo by ligation of integrin alpha5beta1 with the central cell binding domain of fibronectin. Amer. J. Pathology 156:1345-1362.

Kim S; Harris M; Varner J A. (2000) Regulation of integrin alpha vbeta 3-mediated endothelial cell migration and angiogenesis by integrin alpha5beta1 and protein kinase A. J Biol Chem, 275:33920-8.

Koivunen E, Arapl W, Valtanen H, Rainisalo A, Medina O P, Heikkila P, Kantor C, Gahmberg C G, Salo T, Konttinen Y T, Sorsa T, Ruoslahti E, Pasqualini R (1999). Tumor targeting with a selective gelatinase inhibitor Nature Biotechnology 17:768-774.

Kumar R, Yoneda J, Bucana CD, Fidler I J. (1998). Regulation of distinct steps of angiogenesis by different angiogenic molecules. Int. J Oncol. 12:749-757.

Kurdowska A, Fujisawa N, Peterson B, Carr F K, Noble J M, Alden S M, Miller E J, Teodorescu M. (2000) Specific binding of IL-8 to rabbit alpha-macroglobulin modulates IL-8 function in the lung. Inflammation Research 49:591-599.

Lampugnani M-G, Corada M, Caveda L, Breviario F, Ayalon O, Geiger B, Dejana E. (1995). The molecular organization of endothelial cell to cell junctions: differential association of plakoglobin, beta-catenin, and alpha-catenin with vascular endothelial cadherin (VE-cadherin). J. Cell Biol. 129:203-217.

Legrand C; Polette M; Tournier J M; de Bentzmann S; Huet E; Monteau M; Birembaut P. (2001) uPA/plasmin system-mediated MMP-9 activation is implicated in bronchial epithelial cell migration. Experimental Cell Research, 264:326-36.

Li QJ., S. Lu, R. Ye and M. Martins-Green (2000a). Isolation and characterization of a novel CXC chemokine receptor gene. Gene 257:307-317.

Li Q J, and M. Martins-Green (2001). Development and Characterization of a 3D co-Culture System that Mimics Human Skin. Mol. Biol. of the Cell. Suppl. 12: In press.

Li Q J and M. Martins-Green (2001). Molecular mechanisms by which IL-8 stimulates initiation of angiogenesis. Mol. Biol. of the Cell. Suppl. 12: In press.

Li Q J, Vaingankar S, Green H M, Martins-Green M. (1999). Activation of the 9E3/cCAF Chemokine by Phorbol Esters Occurs Via Multiple Signal Transduction Pathways that converge in MEK1/ERK2 and Activate the Elk1 Transcription Factor. J. Biol. Chem. 274:15454-15465.

Li Q J, Vaingankar S, Sladek F, Martins-Green M. (2000b). Novel Nuclear Target for Thrombin: Activation of the Elk-1 Transcription Factor Leads to Chemokine Gene Expression. Blood, Submitted.

Lin T J; Issekutz T B; Marshall J S. 2000. Human mast cells transmigrate through human umbilical vein endothelial monolayers and selectively produce IL-8 in response to stromal cell-derived factor-1 alpha. J Immunol., 165:211-220.

Lin T J; Issekutz T B; Marshall J S. 2001. SDF-1 induces IL-8 production and transendothelial migration of human cord blood-derived mast cells. Internat'l. Archives Allergy and Immunology, 124:142-145.

Luan J, Shattuck-Brandt R, Haghnegahdar H, Owen J D, Strieter R, Burdick M, Nirodi C, Beauchamp D, Johnson K N, Richmond A (1997). Mechanism and biological significance of constitutive expression of MGSA/GRO chemokines in malignant melanoma tumor progression. J Leukocyte Biol 62:588-597.

Lum H; Jaffe H A; Schulz I T; Masood A; RayChaudhury A; Green R D. (1999) Expression of PKA inhibitor (PKI) gene abolishes cAMP-mediated protection to endothelial barrier dysfunction. American Journal of Physiology, 277:C580-8.

Madri J A, Williams S K (1983). Capillary endothelial cell cultures: phenotypic modulation by matrix components. J. Cell Biol 97:153-165.

Martin-Padura I, Lonstaglio S, Scheemann M et al., (1998). Functional adhesion molecule, a novel member of the immunoglobulin superfamily that distributes at intercellular junctions and modulates transmigration. J. Cell Biol 142: 117-127.

Martins-Green, M. (1988). Origin of the dorsal surface of the neural tube by progressive delamination of epidermal ectoderm and neuroepithelium: Implications for neurulation and neural tube defects. Development 103: 687-706.

Martins-Green, M. (1990). Transmission electron microscopy and immunolabelling of tissues for light and electron microscopy. In The Postimplantation Mammalian Embryo: A Practical Approach, A. Copp, ed, pp.127-154, IRL Press, Oxford, UK

Martins-Green M (2000). Dynamics of Cell-ECM interactions with implications for Tissue Engineering. In Principles of Tissue Engineering. pp 33-56 2nd Edition, Eds. R. P. Lanza, W. L. Chick and R. Langer. R. G. Landes Co. (invited).

Martins-Green, M. (2001). 9E3/cCAF. The 9E3/cCAF chemokine. Chapter 10012 in “A Compendium of Cytokines and Other Mediators of Host Defense” (eds. J. Oppenheim, S. Durum), Academic Press Ltd. ftp site: ftp.harcourtbrace.com; directory: /pub/academic_press/ saved/cytokineDB; password: ‘anonymous’.

Martins-Green, M. and Bissell, M. J. (1990). Localization of 9E3/CEF-4 in avian tissues: Expression is absent in Rous sarcoma virus-induced tumors but is stimulated by injury. J. Cell Biol. 110:581-595.

Martins-Green M, Bissell M J (1995). Cell-extracellular matrix interactions in development. Sems. in Dev. Biol. 6:149-159.

Martins-Green, M. and C. A. Erickson (1986). The development of neural tube basal lamina during neurulation and neural crest cell emigration in the trunk of the mouse embryo. J Embryol. Exp. Morph. 98: 219-236.

Martins-Green, M. and C. A. Erickson (1987). Basal lamina is not a barrier to neural crest cell emigration: Documentation by TEM and by immunofluorescent and immunogold labeling. Development 101:517-533.

Martins-Green, M. and C. A. Erickson (1988). Patterns of cholinesterase staining during neural crest cell morphogenesis in mouse and chick embryos. J. Exp. Zool. 247: 62-68.

Martins-Green, M., and Feugate, J. E. (1998). The 9E3/CEF4 gene product is a chemotactic and angiogenic factor that can initiate the wound healing cascade in vivo. Cytokine 10:522-535.

Martins-Green, M. and H. Hanafusa (1997). The 9E3/CEF4 gene and its product the chicken Chemotactic and Angiogenic Factor (cCAF): potential roles in wound healing and tumor development. Cytokines and Growth Factors 8: 229-230.

Martins-Green M., and T. Kelly (1998). The chicken Chemotactic and Angiogenic Factor (cCAF): Its angiogenic properties reside in the C-terminus of the molecule. Cytokine 10:819-829.

Martins-Green, M. and K. T. Tokuyasu (1988). A pre-embedding immunolabeling technique for basal lamina and extracellular matrix molecules. J. Histochem. and Cytochem. 36: 453-458.

Martins-Green, M, A. Aotaki-Keen, L. Hjelmeland and M. J. Bissell (1992). The 9E3 protein: immunolocalization in vivo and evidence for multiple forms in culture. J. Cell Science 101:701-707.

Martins-Green, M., J. L. Bixby, T. Yamamoto, T. Graf and M. Sudol (1999). Tissue Specific Expression of Yrk Kinase: Implications for Differentiation and Inflammation. International J. of Biochem. and Cell Biol. In Press.

Martins-Green, M., Stoeckle, M., Wimberly, S., Hampe, A., Hanafusa, H. (1996). The 9E3/CEF4 Cytokine: Kinetics of secretion, processing by plasmin, and interaction with ECM. Cytokine 8, 448-459.

Martins-Green, M., C. Tilley, R. Schwarz, C. Hatier, and M. J. Bissell (1991). Wound-factor-induced and cell cycle phase-dependent expression of 9E3/CEF4, the avian gro gene. Cell Regulation 2:739-752.

Mazzieri R; Masiero L; Zanetta L; Monea S; Onisto M; Garbisa S; Mignatti P. (1997) Control of type IV collagenase activity by components of the urokinase-plasmin system: a regulatory mechanism with cell-bound reactants. Embo Journal, 16:2319-32.

McCawley L J; Matrisian L M. (2001) Matrix metalloproteinases: they're not just for matrix anymore!. Current Opinion in Cell Biology, 13:534-40.

McDonald D M; Thurston G; Baluk P. (1999) Endothelial gaps as sites for plasma leakage in inflammation. Microcirculation, 6:7-22.

Melkonian, G., C. Li, W. Zheng, P. Talbot and M. Martins-Green (1999). Normal Patterns of Angiogenesis and Extracellular Matrix Deposition in Chick Chorioallantoic Membranes are Disrupted by Mainstream and Sidestream Cigarette Smoke. Toxicology and Applied Pharmacology 163:26-37.

Mignatti P, Rifkin D B, (1996). Plasminogen activators and matrix metalloproteinases in angiogenesis. Enzyme and Protein 49:117-137.

Montesano R, Orci L, Vassalli P. (1983) In vitro rapid organization of endothelial cells into capillary-like networks is promoted by collagen matrices. J. Cell Biol. 97:1648-1652.

Montesano R, Orci L (1985). Tumor-promoting phorbol esters induce angiogenesis in vitro. Cell 42:469-477.

Moore B B, Arenberg D A, Addison C L, Keane M P, Strieter R M. 1998. Tumor angiogenesis is regulated by CXC chemokines. J Lab Clinical Med 132:97-103.

Moser B; Loetscher M; Piali L; Loetscher P. (1998). Lymphocyte responses to chemokines. Internat. Rev Immunol., 16:323-44.

Murdoch C, Monk P N, Finn A (1999). CXC chemokine receptor expression on human endothelial cells. Cytokine 11:704-712.

Murphy G; Gavrilovic J. (1999) Proteolysis and cell migration: creating a path? Current Opinion in Cell Biology, 11:614-21.

Nag S; Picard P; Stewart D J. (2001) Expression of nitric oxide synthases and nitrotyrosine during blood-brain barrier breakdown and repair after cold injury. Laboratory Investigation, 81:41-9.

Nakano M; Atobe Y; Goris RC; Yazama F; Ono M; Sawada H; Kadota T; Funakoshi K; Kishida R. (2000) Ultrastructure of the capillary pericytes and the expression of smooth muscle alpha-actin and desmin in the snake infrared sensory organs. [Erratum In: Anat Rec 2001;263:212]. Anatom. Record 260:299-307.

Navarro P, Caveda L, Breviario F, Mandoteanu I, Lampugnani M-G, and Dejana E. (1995). Catenin-dependent and -independent functions of vascular endothelial cadherin. J. Biol. Chem. 270:30965-30972.

Newman P J. (1999). Switched at birth: a new family for PECAM-1. J. Clinical Inv. 103:5-9.

Owen J D; Strieter R; Burdick M; Haghnegahdar H; Nanney L; Shattuck-Brandt R; Richmond A. (1997). Enhanced tumor-forming capacity for immortalized melanocytes expressing melanoma growth stimulatory activity/growth-regulated cytokine beta and gamma proteins. Int. J Cancer, 26, 73:94-103.

Pear W E, Nolan G P, Scott M L, Baltimore D (1993). Production of high-titer helper-free retroviruses by transient transfection. Proc Natl Acad Sci USA 90:8392-8396.

Proescholdt M A; Heiss J D; Walbridge S; Muhlhauser J; Capogrossi M C; Oldfield E H; Merrill M J. (1999) Vascular endothelial growth factor (VEGF) modulates vascular permeability and inflammation in rat brain. Journal of Neuropathology and Experimental Neurology, 58:613-27.

Raza S L; Cornelius L A. (2000) Matrix metalloproteinases: pro- and anti-angiogenic activities. Journal of Investigative Dermatology. Symposium Proceedings, 5:47-54.

Rennekampff H O; Hansbrough J F; Woods V Jr; Dore C; Kiessig V; Schroder J M. (1997) Role of melanoma growth stimulatory activity (MGSA/gro) on keratinocyte function in wound healing. Archives of Dermatological Research, 289:204-12.

Rival Y, Del Maschio A, Rabiet M J, Dejana E, Duperray A (1996) Inhibition of platelet endothelial cell adhesion molecule-1 synthesis and leukocyte transmigration in endothelial cells by the combined action of TNF-alpha and IFN-gamma. J. Immunol., 157:1233-1241.

Roberts W G, Palade G E (1995). Increased microvascular permeability and endothelial fenestration induced by vascular endothelial growth factor J Cell Science 108:2369-2379.

Rossi F M, Guicherit O M, Spicher A, Kringstein A M, Fatyol K, Blakely B T, Blau H M (1998). Tetracycline-regulatable factors with distinct dimerization domains allow reversible growth inhibition by p16. Nature Genetics 20:389-393.

Rottman J B. (1999) Key role of chemokines and chemokine receptors in inflammation, immunity, neoplasia, and infectious disease. Veterinary Pathology, 36:357-67.

Rupp P A and Little C D. (2001) Integrins in vascular development. Circulation Research 89:566-572.

Sakakibara A, Furuse M, Saitou M, Anddo-Akatsuka Y, and Tsukita S. (1997). Possible involvement of phosphorylation of occludin in tight junction formation. J Cell Biol. 137:1393-1401.

Salcedo R; Wasserman K; Young H A; Grimm M C; Howard O M; Anver M R; Kleinman H K; Murphy W J; Oppenheim J J. (1999). Vascular endothelial growth factor and basic fibroblast growth factor induce expression of CXCR4 on human endothelial cells: In vivo neovascularization induced by stromal-derived factor-1alpha. Amer. J Path., 154:1125-1135.

Salcedo R; Resau J H; Halverson D; Hudson E A; Dambach M; Powell D; Wasserman K; Oppenheim J J. (2000) Differential expression and responsiveness of chemokine receptors (CXCR1-3) by human microvascular endothelial cells and umbilical vein endothelial cells. Faseb Journal, 14:2055-64.

Sanderson R D. (2001) Heparan sulfate proteoglycans in invasion and metastasis. Seminars in Cell and Developmental Biology, 12:89-98.

Schnittler H J. (1998). Structural and functional aspects of intercellular junctions in vascular endothelium. Basic Research in Cardiology, 93 Suppl 3:30-39.

Senger D R, Galli S J, Dvorak A M, Perruzzi C A, Harvey V S, Dvorak H F (1983). Tumor cells secrete a vascular permeability factor that promotes accumulation of ascites fluid. Science 219:983-985.

Sheibani N; Sorenson C M; Frazier W A. (2000) Differential modulation of cadherin-mediated cell-cell adhesion by platelet endothelial cell adhesion molecule-1 isoforms through activatio of extracellular regulated kinases Molecular Biology of the Cell, 11:2793-802.

Spicher A; Guicherit O M; Duret L; Aslanian A; Sanjines E M; Denko N C; Giacci A J; Blau H M. (1998) Highly conserved RNA sequences that are sensors of environmental stress. Molecular and Cellular Biology, 18:7371-82.

Strieter R M; Polverini P J; Arenberg D A; Kunkel S L. (1995a). The role of CXC chemokines as regulators of angiogenesis. Shock, 4:155-60.

Strieter R M; Polverini P J; Arenberg D A; Walz A; Opdenakker G; Van Damme J; Kunkel S L. (1995b). Role of C-X-C chemokines as regulators of angiogenesis in lung cancer. J. Leukocyte Biology, 57:752-62.

Sukow, M A and Douglas, F A. 1997. The Laboratory Rabbit. CRC Press, New York, 145p.

Takai Y; Sasaki T; Matozaki T. (2001) Small GTP-binding proteins. Physiological Reviews, 81:153-208.

Tsukamoto T and Nigam S K, (1999) Cell-cell dissociation upon epithelial cell scattering requires a step mediated by the proteasome. J. Biol. Chem. 274:24579-24584.

Thurston G, Baluk P, Hirata A, McDonald, D M, 1996. Permeability-related changes revealed at endothelial cell borders in inflamed venules by lectin binding, Am. J. Physial. 271:H2547-H2562.

Thurston G, Suri C, Smith K, McClain J, Sato T N, Yancopoulos G D, McDonald D M, 1999. Leakage-resistant blood vessels in mice transgenically overexpressing angiopoetin-1. Science 286:2511-2514.

Vaingankar S, Martins-Green M (1998) Thrombin activation of the 9E3/CEF4 chemokine involves tyrosine kinases including c-src and the EGF receptor. J. Biol Chem 273:5226-5234.

van Hinsbergh V W M, Kookwijk P, and Hanemaaijer R. (1997) Role of fibrin and plasminogen activators in repair-associated angiogenesis: In vitro studies with human endothelial cells. in Regulation of Angiogenesis (Goldberg, I D, and Rosen, E M, eds.) pp 1-8, Birkhäuser Verlag, Basel Switzerland.

Vestweber D. (2000) Molecular mechanisms that control endothelial cell contacts. J Pathology, 190:281-91.

Vu T H; Werb Z. (2000) Matrix metalloproteinases: effectors of development and normal physiology. Genes and Development, 14:2123-33.

Walsh K; Sata M. (1999) Negative regulation of inflammation by Fas ligand expression on the vascular endothelium. Trends in Cardiovascular Medicine, 9:34-41.

Wang H; Keiser J A. (1998) Vascular endothelial growth factor upregulates the expression of matrix metalloproteinases in vascular smooth muscle cells: role of flt-1. Circulation Research, 83:832-40.

Ward S G, Westwick J (1998). Chemokines: understanding their role in T-lymphocyte biology. Biochem J 333:457-470.

White J R; Lee J M; Young P R; Hertzberg R P; Jurewicz A J; Chaikin M A; Widdowson K; Foley J J; Martin L D; Griswold D E; et al. (1998) Identification of a potent, selective non-peptide CXCR2 antagonist that inhibits interleukin-8-induced neutrophil migration. J. Biol Chem. 273:10095-8.

Woolf T M, Jennings C G B, Rebagliati M, Melton D A (1990). The stability, toxicity and effectiveness of unmodified and phosphorotioate antisense oligodeoxynucleotides in Xenopus oocytes and embryos. Nucleic Acids Res. 18:1763-1769.

Wuyts A; Schutyser E; Menten P; Struyf S; D'Haese A; Bult H; Opdenakker G; Proost P; Van Damme J. (2000) Biochemical and biological characterization of neutrophil chemotactic protein, a novel rabbit CXC chemokine from alveolar macrophages. Biochemistry, 39:14549-57.

Yancopoulos G D; Davis S; Gale N W; Rudge J S; Wiegand S J; Holash J. (2000) Vascular-specific growth factors and blood vessel formation. Nature, 407:242-8.

Yi C-F, Gisiewska A, Burtis D, and Geesin G. (2001). Incorporation of Fluorescence Enzyme Substrates in Agarose for in situ Zymography. Analytical Biochemistry 291:27-33.

Youngs S J; Ali S A; Taub D D; Rees R C. (1997) Chemokines induce migrational responses in human breast carcinoma cell lines. International Journal of Cancer, 71:257-66.

Zlotnik A; Yoshie O. (2000) Chemokines: a new classification system and their role in immunity. Immunity, 12:121-7.

Zhou. A. T., Bessalle, R., Bisello, A., Nakamoto, C., Rosenblatt, M., Suva, L. J. and Chorev, M. (1997). Direct mapping of an agonist-binding domain within the parathyroid hormone/parathyroid hormone-related protein receptor by photoaffinity crosslinking. Proc. Natl. Acad. Sci. 94, 3644-3649.

[image]

[image]

[image]

[image]

[image]

[image]

[image]

[image]

[image]

[image]

[image]

[image]

[image]

[image]

[image]

[image]

[image]

[image]

What is claimed is:

1. An artificial tissue comprising a support matrix, microvascular endothelial cells from a first animal, and connective tissue cells from a second animal, wherein the artificial tissue comprises one or more microvessels produced therein.

2. The artificial tissue of claim 1 further comprising epithelial cells from a third animal.

3. The artificial tissue of claim 2 wherein the epithelial cells form a mutlilayered epithelium.

4. The artificial tissue of claims 1 or 2 wherein the first, second and third animals are mammals.

5. The artificial tissue of claim 4 wherein the mammals are selected from the group consisting of primate, mouse, pig, cow, cat, goat, rabbit, rat, guinea pig, hamster, horse, or sheep.

6. The artificial tissue of claim 4 wherein the mammals are humans.

7. The artificial tissue of claims 1 or 2 wherein the first, second and third animals are the same.

8. The artificial tissue of claims 1 or 2 wherein the first, second and third animals are different.

9. The artificial tissue of claim 1 or 2 wherein the support matrix comprises Vitrogen®.

10. The artificial tissue of claim 1 or 2 wherein the microvascular endothelial cells comprise primary human adult lung microvascular cells.

11. The artificial tissue of claim 1 or 2 wherein the connective tissue cells comprise primary human adult dermal fibroblasts.

12. The artificial tissue of claims 2 or 3 wherein the epithelial cells comprise primary human adult keratinocytes.

13. An artificial tissue comprising Vitrogen®, primary human adult lung microvascular cells, and primary human dermal fibroblasts wherein the artificial tissue comprises one or more microvessels produced therein.

14. An artificial tissue comprising Vitrogen®, primary human adult lung microvascular cells, primary human dermal fibroblasts, and primary human keratinocytes wherein the artificial tissue comprises one or more microvessels produced therein.

15. The artificial tissue of claims 1 or 2 wherein the artificial tissue produces one or more compounds selected from the group consisting of laminin, fibronectin, collagen I, collagen III, hyaluronic acid, VEGF 145, VEGF 121, bFGF, IL-8, Syndecan-1, CXCR-1, CXCR-2, a mannose-containing protein, an acetylglucosamine-containing protein, PECAM-1, alpha-SMA, MMP-2, a growth factor receptor, plasminogen activator, mSRA, and CD68.

16. The artificial tissue of claims 1 or 2 wherein the one or more microvessels produce one or more blood cells.

17. The artificial tissue of claims 16 wherein the blood cells comprise mononuclear leukocytes.

18. The artificial tissue of claims 1 or 2 wherein the artificial tissue produces one or more perioendothelial cells.

19. The artificial tissue of claims 1 or 2 wherein the artificial tissue produces an extracellular matrix.

20. The artificial tissue of claims 1 or 2 wherein the artificial tissue is self-maintained.

21. A method for producing an artificial tissue comprising: mixing together a support matrix and connective tissue cells to form a support matrix-connective tissue mixture and forming a culture comprising two layers of support matrix-connective tissue mixture separated by a layer of endothelial cells, wherein said endothelial cells contact inner surfaces of the support matrix-connective tissue mixture layers.

22. The method of claim 21 further comprising plating a layer of epithelial cells on an outer surface of one layer of support matrix-connective tissue mixture.

23. The artificial tissue produced by the method of claim 21 or claim 22.

24. A method for studying a biological process, said method comprising administering a test compound to the artificial tissue of claim 1 or claim 2 and measuring the effect of the test compound on a parameter of the biological process.

25. The artificial tissue of claims 1 or 2 wherein the tissue is maintained in vitro.

26. The artificial tissue of claims 1 or 2 wherein the tissue is a composition suitable for tissue grafting.

27. A method of screening for an agent that inhibits angiogenesis, said method comprising:
a) contacting a biological culture comprising an adhesion polypeptide selected from the group consisting of VE-Cadherin and PE-CAM with a test agent;
b) contacting said biological culture with a chemokine or an angiogenic fragment thereof;
c) detecting the level of phosphorylation of said adhesion polypeptide, wherein: an increase in the level of phosphorylation, as compared to said level in a biological culture of the same type contacted with a smaller amount of the test agent, indicates that the test agent inhibits anglogenesis.

28. The screening method of claim 27 wherein said method additionally comprises recording any test agent that reduces the level of phosphorylation in a database of agents that inhibit angiogenesis.

29. The screening method of claim 27 wherein said smaller amount of the test agent is no test agent.

30. The screening method of claim 27 wherein the chemokine is a CXC chemokine.

31. The screening method of claim 30 wherein the chemokine is interleukin-8 (IL-8).

32. The screening method of claim 30 wherein said biological culture is in vitro.

33. The screening method of claim 30 wherein the biological culture is the chorioallantoic membrane (CAM).

34. A method of prescreening for an agent that inhibits angiogenesis, said method comprising:
a) contacting an adhesion polypeptide selected from the group consisting of VE-Cadherin and PE-CAM with a test agent; and
b) detecting specific binding of the test agent to the adhesion polypeptide.

35. A method of screening for an agent that inhibits angiogenesis, said method comprising:
a) contacting a biological culture comprising MMP-9 with a test agent;
b) contacting said biological culture with a chemokine or an angiogenic fragment thereof,
c) detecting the level of MMP-9 activity, wherein a decrease in the level of MMP-9 activity, as compared to said level in a biological culture of the same type contacted with a smaller amount of the test agent, indicates that the test agent inhibits angiogenesis.

36. The screening method of claim 35 wherein said method additionally comprises recording any test agent that reduces the level of MMP-9 activity in a database of agents that inhibit angiogenesis.

37. The screening method of claim 35 wherein said smaller amount of the test agent is no test agent.

38. The screening method of claim 35 wherein the chemokine is a CXC chemokine.

39. The screening method of claim 38 wherein the chemokine is interleukin-8 (IL-8).

40. The screening method of claim 38 wherein said biological culture is in vitro.

41. The screening method of claim 38 wherein the biological culture is the artificial tissue of claim 1.

42. A method of prescreening for an agent that inhibits angiogenesis, said method comprising:
a) contacting MMP-9 with a test agent; and
b) detecting specific binding of the test agent to MMP-9.
